(19) United States
(12) Patent Application Publication
(10) Pub. No.: US 2022/0192854 A1
(43) Pub. Date: Jun. 23, 2022

(54) PYLORIC DEVICE

(71) Applicant: Boston Scientific Scimed, Inc., Maple Grove, MN (US)

(72) Inventors: Ryan D. Lynch, Roscommon (IE); James Quinn, Galway (IE); Darren G. Curran, Galway (IE); Gerard Duignan, Galway (IE)

(21) Appl. No.: 17/554,653

(22) Filed: Dec. 17, 2021

Related U.S. Application Data
(60) Provisional application No. 63/129,224, filed on Dec. 22, 2020.

(51) Int. Cl. A61F 5/00

(52) U.S. Cl. CPC A61F 5/0079; A61F 5/0089

(57) ABSTRACT

The present disclosure is related to medical devices, stents, occlusion devices, and the like, to occlude, limit, or otherwise facilitate a regulated fluid flow between body lumens. A medical device may be a pyloric occlusion device with first and second configurations. In the second configuration, the elongate body may comprise a first retention member, a second retention member, and a cylindrical saddle region configured to span a pyloric sphincter. The first retention member and the second retention member each include an inner wall, an outwardly circumferential wall, and an outer wall, a first interface connecting the inner wall and the outwardly circumferential wall, and a second interface connecting the outwardly circumferential wall and the outer wall. At least one of the outwardly circumferential wall or the interface of either the first retention member or the second retention member is configured to appose a pyloric antrum wall.

### PRIORITY

The present application is a non-provisional of, and claims the benefit of priority under 35 U.S.C. § 119 to, U.S. Provisional Application Ser. No. 63/129,224, filed Dec. 22, 2020, the disclosure of which is hereby incorporated herein by reference in its entirety for all purposes.

### FIELD

The present disclosure relates generally to the field of medical devices and methods for partially, temporarily, intermittently, and/or fully obstructing a body lumen. In particular, medical devices, systems, and methods are directed towards pyloric occlusion during gastrojejunostomy procedures.

### BACKGROUND

Various medical approaches are used for treating bariatric or metabolic diseases, such as diet, medication, and surgical procedures. Surgical procedures such as bariatric surgery, e.g., to restrict a portion of a stomach and/or bypass portions of the intestine, may be the only option for some patients.

It is therefore desirable to provide a successful and minimally invasive alternative to existing approaches for treating bariatric or metabolic diseases.

With the above considerations in mind, a variety of advantageous medical outcomes may be realized by the devices and/or methods of the present disclosure.

### SUMMARY

According to at least one aspect of the present disclosure, a pyloric occlusion device may comprise an elongate body having a first configuration and a second configuration. In the second configuration, the elongate body may comprise a first retention member, a second retention member, and a cylindrical saddle region extending therebetween. The first retention member and the second retention member may each or individually include an inner wall, an outwardly circumferential wall forming a cylindrical portion, and an outer wall. A first interface may connect the cylindrical saddle region and the inner wall. A second interface may connect the inner wall and the outwardly circumferential wall. A third interface may connect the outwardly circumferential wall and the outer wall. In the second configuration, the first and the second retention members may be expanded to have greater diameters than in the first configuration. In the second configuration, at least one of the outwardly circumferential wall, the second interface, or the third interface of either the first retention member or the second retention member may be configured to appose a pyloric antrum wall. The cylindrical saddle region may be configured to span a pyloric sphincter.

According to the above and other embodiments of the present disclosure, at least a portion of the outer wall may be covered and one or both of the first and second interfaces may be uncovered. One or both of the first and second interfaces may be covered and at least a portion of the outer wall may be uncovered on at least one of the first or second retention members. The first retention member, the second retention member, or both may comprise a lip. The lip may comprise a different diameter than a cylindrical saddle region diameter. Each of the first and second retention members may extend along a length at least 75% greater than a length of the cylindrical saddle region. The first interface of one or both of the first or second retention members may comprise a corner or crease. The second interface, the third interface, or both of one of the first or second retention members may comprise a corner or crease. The corner or crease may be configured to straighten in order to allow the elongate body to move from the second configuration to the first configuration. The inner wall, the outer wall, or both of the first retention member may comprise a substantially straight portion. The inner wall and the outer wall of at least one of the first or second retention members may be substantially parallel. The inner wall and the outer wall of at least one of the first or second retention members may be non-parallel. The outer wall, the inner wall, or both of the first retention member may comprise a convex portion bending toward a vertical center plane of the first retention member. The outer wall, the inner wall, or both of the first retention member may comprise a concave portion bending away from a vertical center plane of the first retention member. A diameter of the first retention member, a diameter of the second retention member, or both may be 300-600% a diameter of the cylindrical saddle region. The cylindrical saddle region may define a lumen extending longitudinally therethrough. The cylindrical saddle region may comprise a cover adjacent to the inner wall of the first retention member, the second retention member, or both. The cylindrical saddle region may define an outer surface of the elongate body extending a full length between the first and the second retention members. The pyloric occlusion device may comprise at least one cap.

In another aspect of the present disclosure, a stent may comprise an elongate body having a first configuration and a second configuration. In the second configuration, the elongate body may comprise a saddle region and a gastric extension. The gastric extension may have an inner wall, an outwardly circumferential wall, and an outer wall. A first interface may connect the inner wall and the outwardly circumferential wall. A second interface may connect the outwardly circumferential wall and the outer wall. One or both of the first interface and the second interface may include a concave portion and a convex portion. The outwardly circumferential wall may define a cylindrical portion with a length sufficient to encourage tissue ingrowth therealong.

According to the above and other aspects of the present disclosure, one or both of the first interface and the second interface may include a substantially straight portion. At least a portion of the outer wall may be covered and one or both of the first and second interfaces may be uncovered. One or both of the first and second interfaces may be covered and at least a portion of the outer wall may be uncovered. The first interface, the second interface, or both may comprise a corner or crease. The cylindrical portion may extend along a length that is at least 75% of a length of the saddle region. A diameter of the inner wall may be at least 28 mm. A diameter of the saddle region may be less than 5 mm. The cylindrical saddle region may define an outer surface of the elongate body extending a full length between the first and the second retention members.

In yet another aspect of the present disclosure, a pyloric occlusion device may comprise an elongate body having a constrained configuration and an unconstrained configuration. In the unconstrained configuration, the elongate body may comprise a first retention member, a second retention member, and a saddle region extending therebetween. The saddle region may be configured to span a pyloric sphincter. One of the first retention member or the second retention member may comprise an inner wall, an outwardly circumferential wall, and an interface between the inner wall and the outwardly circumferential wall. At least one of the outwardly circumferential wall or the interface may be configured to appose a pyloric antrum wall. The outwardly circumferential wall may not conform to a shape of the pyloric antrum wall.

According to the above and other aspects of the present disclosure, the interface may comprise a corner or crease. The pyloric occlusion device may comprise an axially outward surface and a second interface between the outer wall and the outwardly circumferential wall. At least a portion of the outer wall may be covered and one or both of the interface and second interface may be uncovered on at least one of the first or second retention members. One or both of the interface and second interface may be covered and at least a portion of the outer wall may be uncovered on at least one of the first or second retention members.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIG. 1 illustrates a side view of an occlusion device according to one or more embodiments of the present disclosure.

FIG. 2 illustrates a side view of an additional occlusion device according to one or more embodiments of the present disclosure.

FIG. 3 illustrates a side view of an additional occlusion device according to one or more embodiments of the present disclosure.

FIG. 4 illustrates a side view of regions and interfaces of occlusion devices of the present disclosure which may comprise a covering.

FIGS. 5A-5B illustrate side views of an occlusion device deployment across first and second body lumens, according to at least one embodiment of the present disclosure.

FIGS. 6A-6F illustrate side and end views of occlusion devices according to various embodiments of the present disclosure.

FIG. 7A-7C illustrate end view of caps for occlusion devices, according to various embodiments of the present disclosure.

FIGS. 8A-L illustrate cross-sectional profiles of retention member walls, according to various embodiments of the present disclosure.

### DETAILED DESCRIPTION

The present disclosure is related to medical devices, stents, occlusion devices, and the like, which include retention features such that are configured to lodge, position, or otherwise maintain the medical device across body lumens. Many embodiments may occlude, limit, or otherwise facilitate a regulated fluid flow between body lumens.

For example, natural orifice transluminal endoscopic surgery (NOTES) procedures may be advantageous over other types of bariatric procedures, such as gastric procedures, by enabling a redirection of flow (e.g., chyme or other gastric flow) via an anastomosis (e.g., created between the stomach and a jejunal loop of small bowel in the jejunum). However, redirection of flow may necessitate an occlusion of a natural flow path in favor of the surgically introduced alternative path.

Devices, systems, and methods described herein may assist in fluid flow path occlusion, and in particular occlusion of gastric flow into the duodenum, which may limit digestion of food, liquid, and other nutrients until further down the GI tract. Many embodiments may relate to endoscopic ultrasound procedures. In some embodiments, a pyloric closure or occlusion device may be reversible, e.g., a medical professional, physician, and/or automated system may be able to deliver and/or remove the device endoscopically. Although the devices, systems, and methods are described herein with respect to a gastrointestinal system, it will be understood that illustrative embodiments and/or elements and/or features of devices, systems, and methods in accordance with various principles of the present disclosure may be advantageous for use in any other procedures and/or anatomy, for deployment of an occlusion device to prevent movement of material.

In embodiments of the present disclosure, devices may comprise retention members at opposing ends of a saddle region of a stent, which may include flanges, bulbs, or the like. At least one retention member of presently described embodiments may be configured to be disposed in a pyloric antrum and to interface with/appose a wall thereof.

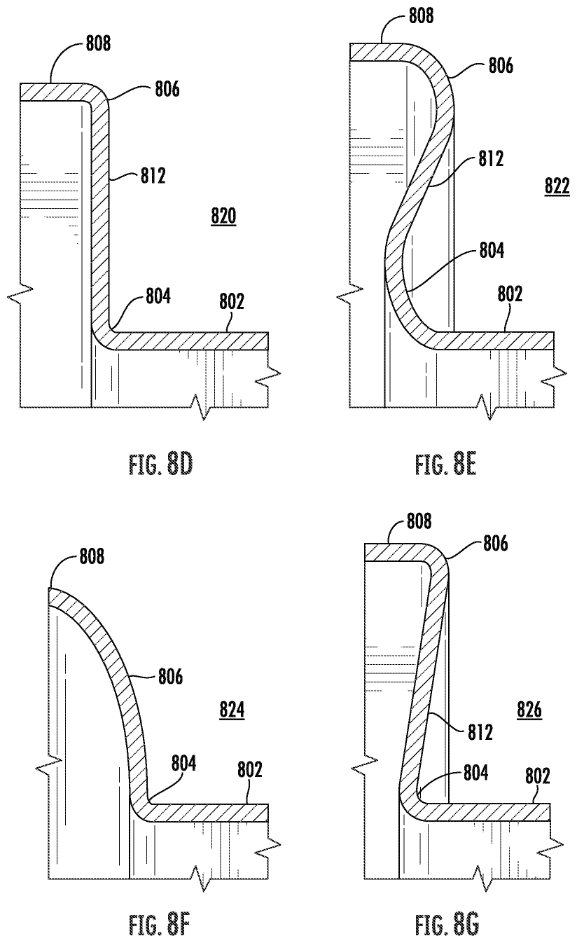

Several existing devices include retention members which may appose a pyloric sphincter at an inner wall of a retention member, the inner wall facing a saddle region. For example, inner surface 745 or inner surface 750 of the occlusion device 700 of FIG. 7, or inner surface 1045 of occlusion device 1000 of FIG. 10, of United States Patent Application Publication No. 2019/0298559 may appose a pyloric sphincter. United States Patent Application Publication No. 2019/0298559, published on Oct. 3, 2019, corresponds to currently pending U.S. application Ser. No. 16/361,772, which was filed on Mar. 22, 2019 and is presently incorporated by reference herein in its entirety for all purposes. Existing devices may focus on apposition of the pyloric sphincter, for example, due to the motility of the stomach and the relative stability of the pyloric sphincter.

Existing devices may include duodenal extensions, which may provide a holding force in the duodenum.

In accordance with various principles of the present disclosure, retention of occlusion devices positioned across the pyloric sphincter may benefit from retention members placed at least partially in the stomach which have a longitudinal length sufficient to encourage ingrowth of tissue into the retention members and/or to appose a circumferential tissue of the pyloric antrum despite motility of the stomach. Retention of occlusion devices positioned across the pyloric sphincter may benefit from retention members placed in the stomach which have a longitudinal length sufficient to encourage seating of the retention members and/or their stability within the pyloric antrum and/or stomach, particularly extending from the pyloric sphincter at least into the pyloric antrum.

Without wishing to be bound by any theory, it is believed that a gastric extension of an occlusion device may encourage a surprising degree of tissue ingrowth of the pyloric antrum tissue into an exposed outwardly circumferential wall (e.g., uncovered surface) of the gastric extension, thereby reducing migration risk and/or improving retention of the deployed device. In particular, a gastric extension of a medical device as presently disclosed may interact with the narrowing geometry of the pyloric antrum towards the pyloric sphincter despite motility of the stomach. Accordingly, medical devices of the present disclosure may have multiple retention members each with an outwardly circumferential wall configured to appose a tissue defining a lumen in which the respective retention member is placed (e.g., including an outwardly circumferential wall configured to engage with a wall of a pyloric antrum).

In accordance with at least some principles of the present disclosure, retention members of occlusion devices described herein may be placed in the stomach which extend longitudinally into the stomach, but which do not encourage tissue ingrowth therealong. Without wishing to be bound by any theory, it is believed that a gastric extension of an occlusion device may provide a contact surface cushioning interaction of the occlusion device with the stomach, and/or provide a surface frictionally resistant to relative sliding of the gastric extension. Accordingly, embodiments described herein may present reduced migration risk and/or improved retention over alternative designs. In particular, a gastric extension of a medical device as presently disclosed may interact with (e.g., partially or fully appose tissue of) the narrowing geometry of the pyloric antrum towards the pyloric sphincter despite motility of the stomach.

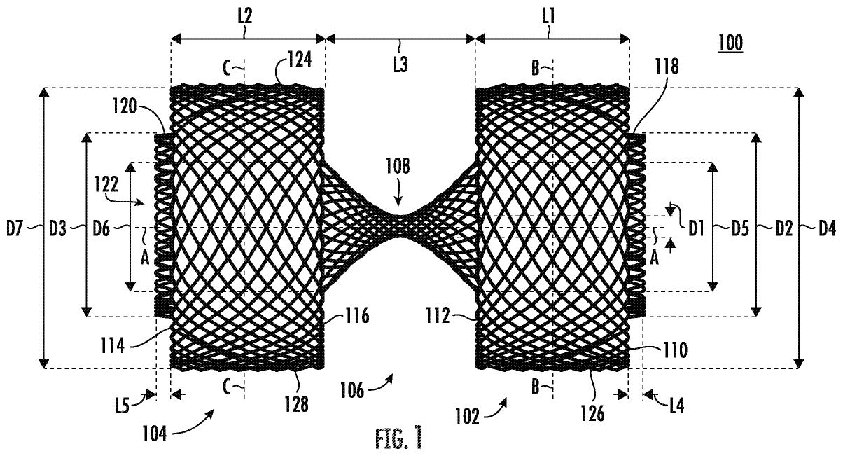

Conventional devices configured to appose a pyloric sphincter have additionally or alternatively sought to extend the apposing surface to approximate a surface of the pyloric antrum. For example, pyloric obesity valve 12 of FIG. 1, of U.S. Pat. No. 8,840,679, hereby incorporated by reference in its entirety, may approximate a pyloric antrum surface. Approximation of the surface of the pyloric antrum may increase an exposed surface of a device to the tissue, particularly in the motile environment of the stomach. According to various principles of the present disclosure, a gastric extension of an occlusion device characterized by a length outstretching into the stomach may accommodate greater tissue ingrowth along the gastric extension, surprisingly in spite of the known motility of the stomach. In particular, retention members which do not conform to or match the surface of the pyloric antrum may encourage retention of corresponding medical devices. Without wishing to be bound by any theory, it is believed that surfaces extending into the stomach, and in many embodiments which do not conform to the surface of the pyloric antrum, may somewhat traumatically engage with the pyloric antrum to trigger a growth response of the tissue thereof in spite of, and potentially particularly, as a result of the motility of the stomach. Thus, tissue may have increased ingrowth into a medical device comprising an outwardly circumferential wall of a gastric extension as opposed to alternative configurations.

Accordingly, presently disclosed medical devices include at least portions of retention members which extend through the pylorus towards the gastric body, which may not conform to the pyloric antrum surface. For example, an interface between an axially inner wall or an axially outer wall and an outwardly circumferential wall of present embodiments may include a curve, angle, or juxtaposition which does not closely mirror a tissue surface, but which continues roughly or approximately to the tissue surface so as to accommodate ingrowth from the tissue surface. In some embodiments, a retention member diameter may be less than a diameter of the corresponding body lumen in which the retention member is configured to be placed. For example, a gastric extension may include a diameter less than a corresponding diameter of the pyloric antrum. In various embodiments, a retention member diameter may be greater than a diameter of the corresponding body lumen in which the retention member is configured to be placed. For example, gastric extensions may include diameters greater than a corresponding diameter of the pyloric antrum.

In various embodiments, medical devices as disclosed herein include saddle regions configured to extend through a pyloric sphincter, and/or retention members configured to extend into a duodenum, away from the pylorus. Medical devices may be configured to encourage and/or otherwise facilitate tissue ingrowth along portions disposed in a duodenum and/or pyloric sphincter. Saddle regions may define lumens with internal diameters sufficient to allow flow-through of residual fluid but not of substantive food particles. In various embodiments, saddle regions may not include lumens, or may include lumens sufficient for a guidewire passage during delivery but not for sustained fluid flow therethrough.

According to various embodiments, occlusion device retention members may be double-walled and/or include one or more inner and outer walls (e.g., surfaces), which may extend generally transverse to a longitudinal axis extending through a saddle device of the occlusion device. As used herein, an “inner wall” may refer to a wall of a retention member positioned on a side of the retention member nearest a saddle region. An “outer wall” may refer to a wall of a retention member positioned on the side of the retention member farthest from the saddle region (e.g., nearest an end of the stent).

In many embodiments, inner and outer walls may be non-perpendicular, for example, with respect to a plane containing a longitudinal axis of the medical device. Non-perpendicular surfaces may include one or more substantially straight configurations, one or more concave portions, and/or one or more convex portions. Concave portions and/or convex portions may be defined with respect to an outside of a medical device and a positioning of a saddle region on the medical device; concave portions may bend away from an interior lumen of the medical device as viewed from a perspective of a saddle region, and convex portions may bend towards an interior lumen of the medical device as viewed from the saddle region.

Concave and/or convex portions of retention members described herein may individually comprise at least one acute and/or obtuse interior angle, individual length, individual radius of curvature, or other geometrical feature. Such configurations of non-perpendicular surface may reduce migration of the medical device with respect to the tissue(s) between the first and second body lumens, when compared, for example, to a corresponding retention member with only perpendicular surfaces. Additionally, or alternatively, retention members with one or more non-perpendicular surfaces, may be configured to provide more control over the resistance of the device being pulled out of its intended placement once deployed, e.g., resulting in higher pull-out forces as compared to a corresponding retention member with surfaces perpendicular to plane containing a longitudinal axis of the medical device (e.g., perpendicular surfaces). Without wishing to be bound by any theory, concave and/or convex surfaces of retention members may contribute to higher pull-out forces of medical devices over alternative designs by correspondingly increasing an amount of force necessary to flatten out or otherwise contract or compress the retention member sufficiently to dislodge or disposition it from within a deployed position (e.g., dislodgement through a pyloric sphincter).

Retention members of devices disclosed herein may comprise one or more curved portions, substantially straight portions, angled portions, or any combination thereof, wherein each portion may comprise an equal or a different length, angle, interior radius of curvature, directionality, interior angle, or other feature with respect to another portion. Surfaces of first retention members and second retention members may be the same or different. For example, each end of a medical device may be designed to improve the strength (e.g., resistance to pull-out or retentive strength, or resistance to radial compression or radial strength) of a medical device and provide a desired amount of linear apposing force when placed across tissue planes. Retention member shapes may comprise one or more rolls and/or structural folds, for example, to create a double-walled flange structure. In various embodiments, retention member shapes may comprise a plurality of inflection points, wherein an inflection point may be a point of a curve at which a change in direction of curvature occurs.

In various embodiments, one or more non-perpendicular surfaces of retention members (e.g., with respect to planes including one or both of axes of a saddle region and/or a lip as described herein) may also be configured to interact more traumatically or less traumatically with at least one tissue wall of the first and second body lumens (e.g., configuring a retention member wall with a point of tissue contact having less surface area), as compared to conventional designs. It will be appreciated that aggressively injurious medical device contact with tissue may cause discomfort and/or increase a risk of infection for a patient. However, insufficient interaction of medical devices with surrounding tissue may result in medical device migration. In several presently considered embodiments, medical device contact with surrounding tissue, then, may be configured to cause minor abrasions to and/or sufficiently trigger a tissue growth response so as to lead to ingrowth of the tissue into a medical device surface, thereby securing the medical device with respect to the tissue.

A medical device, such as an occlusion device, may be formed of one or more filaments and/or surfaces. In various embodiments, one or multiple wires, braids of one or multiple wires, polymeric filaments, sheets, or a combination thereof may form a medical device. For example, a length of braid of one or multiple wires may form a medical device. A medical device may include one or more structural elements such as a strut, hoop, mesh, tessellating cell, or other unit. In many embodiments, a medical device may comprise a mesh, weave, and/or knit surface. A medical device may be formed, in various embodiments, of a shape memory material, such as Nitinol or a shape memory polymer, which may be structured as a filament, sheet, or other shape. For the sake of simplicity, the present disclosure may refer to a material of a medical device as a woven filament (e.g., a woven filament 124), but embodiments may alternatively and/or additionally comprise other materials and/or configurations of filament. In various embodiments, medical devices as described herein may be wire stents.

Some embodiments may comprise a covering, coating, or other membrane, which may inhibit tissue growth and/or minimize fluid leakage from within and/or without the medical device. Various embodiments of medical devices described herein may include a full or partial covering, coating, or other membrane over an interior, over an exterior of the devices, extending between structural elements, or any combination thereof. For example, a covering, coating, or other membrane may comprise silicone, a polymer, or a combination thereof. For example, a cover may comprise polyurethane, polytetrafluoroethylene, expanded polytetrafluoroethylene, polyvinylidene fluoride, an aromatic polycarbonate-based thermoplastic urethane, and/or other like materials. A cover may be applied by dip coating, roll coating, painting, spraying, other known disposition method, or a combination thereof.

At least one covering, coating, or other membrane may extend fully or partially over medical devices as described herein. For example, a first retention member, a second retention member, a saddle region extending between a first retention member and a second retention member, or a combination thereof, may comprise a solid covering, a porous covering, or other configuration of covering. In some embodiments, a circumferential covering or coating may be applied to cover the full length of the stent, or a partial length of the stent. For example, a partial coating may cover the full length of the saddle region, but not the retention members. Embodiments are not limited herein.

The present disclosure is not limited to the particular embodiments described. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices (e.g., occlusion devices, stents, etc.) and methods for controlling drainage of (or access to) the digestive system, it should be appreciated that such medical devices and methods may be used in a variety of medical procedures to establish and/or maintain a temporary or permanent controlled flow passage between or drainage passage from a variety of body organs, ducts, lumens, vessels, fistulas, and spaces (e.g., the dermis, stomach, duodenum, gallbladder, bladder, kidneys, heart, abscesses, valves, ducts, etc.). For example, congenital conditions and/or conditions resulting from other medical procedures may be treated. The devices can be inserted via different access points and approaches, e.g., percutaneously, endoscopically, laparoscopically, or some combination. Various stents described are self-expanding stents, but other embodiments where the stent is expandable by other means, for example, a balloon catheter, may be possible. Moreover, such medical devices may not be limited to occlusion, but in some embodiments may facilitate drainage, access to organs, vessels, or body lumens for other purposes, such as creating a path to divert or bypass fluids or solids from one location to another, removing obstructions, and/or delivering therapy, including non-invasive manipulation of the tissue within the organ, and/or the introduction of pharmacological agents via the open flow passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components, and/or groups thereof.

As used herein, the term “distal” refers to the end farthest away from the medical professional, physician, or automated system when introducing a device into a patient, while the term “proximal” refers to the end closest to the medical professional, physician, or automated system when introducing a device into a patient.

As used herein, the term “cylindrical” refers to a longitudinally extending circumferential surface, which comprises a substantially constant diameter along its entire longitudinal length, although non-circular cross-sections are not excluded.

A used herein, the term “lumen” may refer to a cavity, vessel, or other space defined by at least one wall. For example, a body lumen may be a stomach, a duodenum, a pyloric antrum, or the like.

Unless otherwise stated, incremental values and/or ranges described herein will be understood to be inclusive and to include iterative smaller ranges therein with endpoints at intervals of 0.1 for quantitative ranges or of 1% for qualitative ranges. For example, a range described as “0.0-2.0 mm” will be understood to comprise included ranges of 0.0-1.9 mm, 0.0-1.8 mm, 0.0-0.7 mm, etc., as well as 0.1-2.0 mm, 0.2-2.0 mm, 0.3-2.0 mm, etc., and any combination of end points covered thereby (e.g., 0.1-1.9 mm, 0.2-1.7 mm, etc.). In another example, a range described as 100-700% will be understood to include 100-699%, 100-698%, 100-687%, etc., 101-700%, 102-700%, 103-700%, etc., 101-699%, 102-698%, 103-697%, etc.

It will be understood that various embodiments described herein may include one or more features discussed herein. For example, combinations of concepts relating to any aspects of the present disclosure are currently contemplated.

FIG. 1 illustrates a side view of an occlusion device 100 (e.g., stent) according to various embodiments herein. In many embodiments, occlusion device 100 may be configured to move between a first configuration (e.g., constrained configuration, delivery configuration, compressed configuration, etc.) and a second configuration (e.g., unconstrained configuration, deployed configuration, expanded configuration, etc.). Occlusion device 100 in the second configuration comprises an expanded first retention member 102, an expanded second retention member 104, and a saddle region 106 extending therebetween along a longitudinal axis A-A. In various embodiments, first retention member 102, second retention member 104, and saddle region 106 may define a lumen 122. In particular, saddle region 106 comprises a narrow portion 108 to limit flow through lumen 122 by restricting a minimal diameter “D1” of lumen 122. Embodiments are not limited herein.

First retention member 102 can include an outer wall 110 (e.g., proximal wall) and/or an inner wall 112 (e.g., distal wall). Second retention member 104 can include an outer wall 114 (e.g., distal wall) and/or an inner wall 116 (e.g., proximal wall).

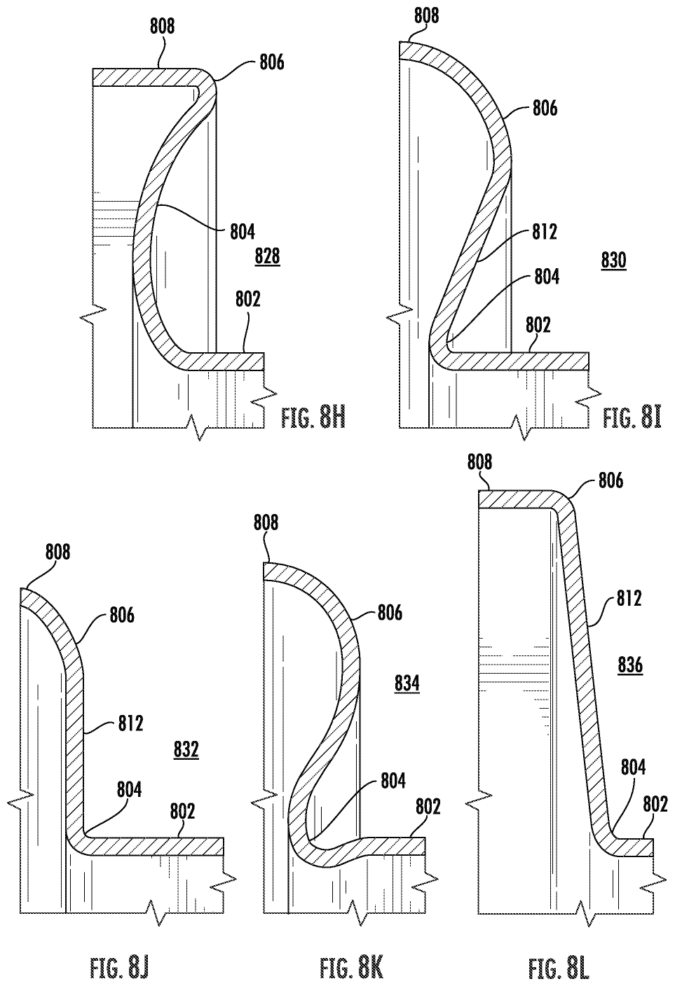

Outer wall 110 may be connected to (e.g., continuously formed with) inner wall 112 via outwardly circumferential wall 126 of length “L1” along longitudinal axis A-A. Similarly, outer wall 114 may be connected to inner wall 116 via outwardly circumferential wall 128 of length “L2” along longitudinal axis A-A. In many embodiments, L1 and L2 may be the same, but other embodiments include first and second retention members 102, 104 of different lengths L1, L2. Occlusion device 100 may include inner walls 112, 116 and outer walls 110, 114 that are substantially perpendicular to longitudinal axis A-A, and as such, L1 and L2 simultaneously describe lengths of first and second retention members 102, 104 as well as respective outwardly circumferential walls 126, 128. However, when at least one of inner walls 112, 116 and outer walls 110, 114 is not perpendicular to longitudinal axis A-A, first and second retention members 102, 104 may have respectively different lengths than outwardly circumferential walls 126, 128. For example, any or each of inner walls 112, 116 or outer walls 110, 114 may comprise one of the illustrative surfaces of FIGS. 8A-8L.

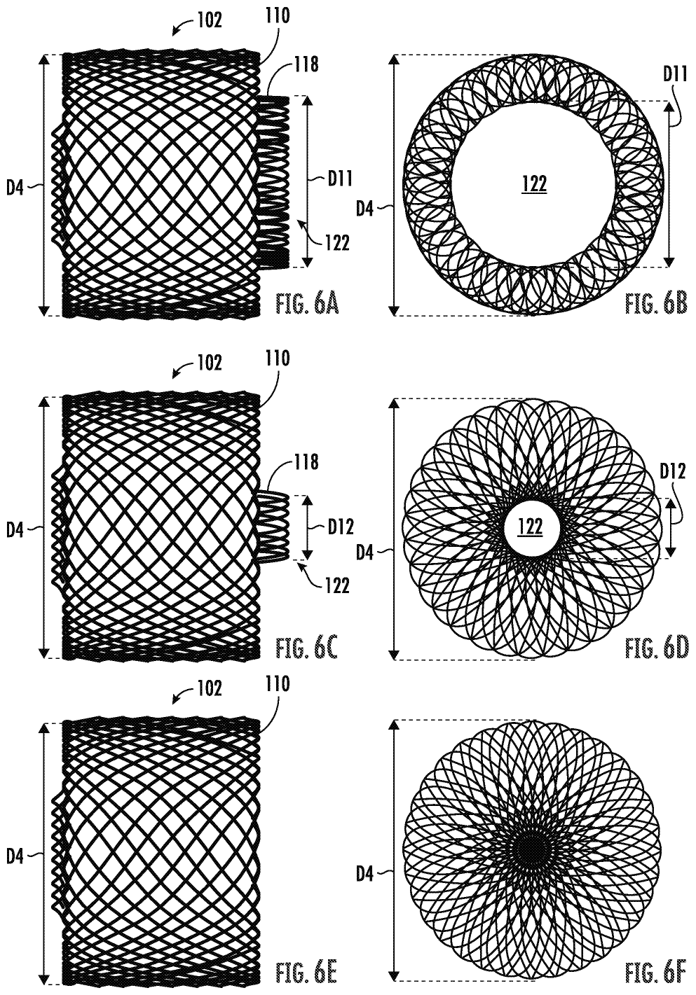

Outwardly circumferential walls 126, 128 may be substantially parallel to longitudinal axis A-A and have respective diameters “D4” and “D7.” Accordingly, each of first and second retention members 102, 104 may include approximately cylindrical portions. L1 and/or L2 comprising a length great enough to make first and second retention members 102, 104 bulbous, and/or sufficient to enable tissue ingrowth along outwardly circumferential walls 126, 128 (e.g., extended flanges). In some embodiments (not illustrated), outwardly circumferential walls 126 and 128 may have different diameters D4 and D7. In various embodiments, D4 and/or D7 may be great enough so as to occlude a respective first or second retention member 102, 104 from passing through a tissue orifice spanned by saddle region 106 (e.g., a tissue orifice 506 as illustrated in FIG. 5B).

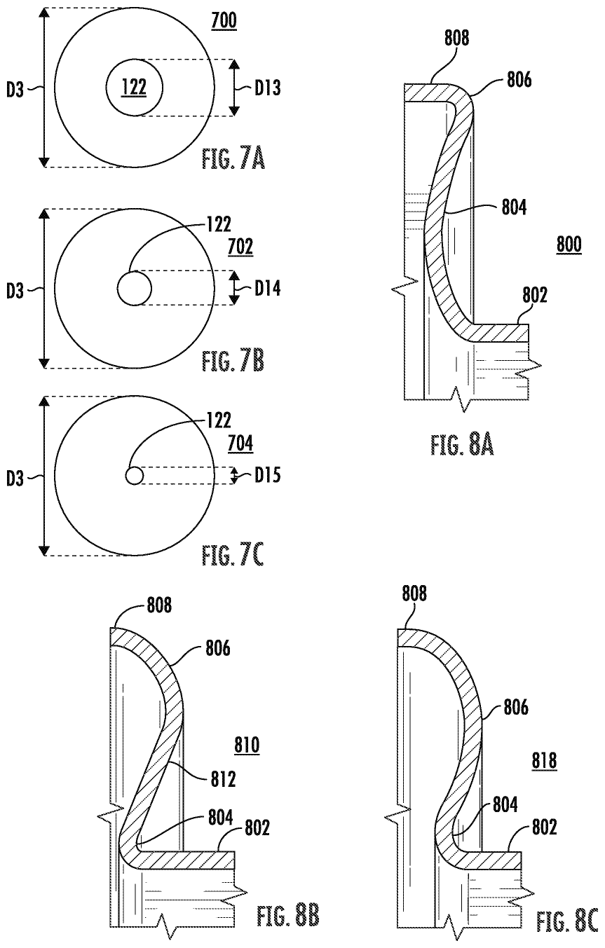

In some embodiments, first retention member 102 may comprise a lip 118 of length “L4” along longitudinal axis A-A. Second retention member 104 may comprise a lip 120 of length “L5” along longitudinal axis A-A. Lips 118, 120 may be axial extensions from a respective first or second retention member 102, 104. Lips 118, 120 may have respective diameters “D2” and “D3,” which may be the same or different. While lips 118, 120 are illustrated with constant diameters D2, D3, it will be understood that lips 118, 120 may have varying diameters D2, D3 along their lengths L4, L5, for example, increasing and/or decreasing along lengths L4, L5. Lumen 122 can extend through lip 118, 120. Lip 118, 120 may provide various benefits, which may include providing a location for grasping during placement and/or removal of occlusion device 100. Additionally, or alternatively, lip 118, 120 may provide an additional geometrical feature which may support a respective outer wall 110, 114, and/or which may resist deformation of the respective retention member 102, 104. For example, deformation of first or second retention member 102, 104 may require deformation of corresponding lip 118, 120. Accordingly, for some situations, lip 118, 120 may increase a retentive strength of occlusion device 100 above alternative configurations. Some embodiments may not comprise a lip 118, 120, for example, rather having a flat end surface as shown of outer wall 114 in the side view of FIG. 6E.

Returning to FIG. 1, saddle region 106 may extend between first and second retention members 102, 104, and in many embodiments may be continuously formed therewith, for example, of woven filament 124. Saddle region 106 can comprise a varying diameter, connecting to first retention member 102 at a diameter “D5” and to second retention member 104 at a diameter “D6,” but having a smaller diameter D1 at narrow portion 108. Narrow portion 108 may be formed by applying a twist to saddle region 106 or by forming saddle region 106 around a mandrel with a varying surface (not illustrated). Alternatively, narrow portion 108 may be formed by applying a tie, sleeve, or other constraint (not illustrated). Lumen 122 may extend through saddle region 106, but in many cases, narrow portion 108 may restrict or reduce flow through lumen 122, for example, by virtue of its relatively decreased diameter D1. Without wishing to be bound by any theory, it is believed that limited fluid flow through the pyloric sphincter to the duodenum may prevent an improper starvation response in a patient. Lumen 122 may accordingly, in many embodiments, allow limited fluid flow through the occlusion device 100. Narrow portion 108 is illustrated as being longitudinally centered along saddle region 106. However, it will be understood that narrow portion 108 may alternatively be uncentered along saddle region 106, for example, forming a taper where one side of saddle region 106 comprises diameter D1 and wherein a saddle region diameter increases along a longitudinal length of saddle region 106 (not shown). In various embodiments, particularly in which saddle region 106 of occlusion device 100 is configured to be disposed across a pyloric sphincter (not shown), D1 may be less than 5 mm, and in particular, 3-4 mm, 1-2 mm, or 0-1 mm.

Various embodiments may include relative dimensions between elements thereof which may be configured to provide one or more of the benefits as described herein. In various embodiments, D4 and/or D7 may be 100-700% the magnitude of D1, D5, or D6. In some embodiments, D4 and/or D7 may alternatively by 300-600%, or 100-500%, or 300-400% the magnitude of D1, D5, or D6. In many embodiments, D4 and/or D7 may be equal or approximately equal to L3 (e.g., within 5% of L3). In various embodiments, D4 and/or D7 may be 20-200% greater than L3, 75-125% greater than L3, 100-150% greater than L3, or 50-75% greater than L3. A larger diameter of D4 and/or D7 with respect to D1, D5, and/or D6 may increase a surface area of contact of the respective inner wall 112, 116 with a pyloric antrum with respect to conventional devices, a smaller diameter of D1, D5, and/or D6 with respect to D4 and/or D7 may decrease a fluid flow rate through lumen 122 with respect to conventional devices while maintaining contact of an inner wall 112 or inner wall 116 with a pyloric antrum, or both. According to various embodiments, and particularly when one of first or second retention member 102, 104 is configured to be positioned in a pylorus, the corresponding length D4 or D7 may be at least 15 mm, 20 mm, 25 mm, 27 mm, 28 mm, 29 mm, 30 mm, 31 mm, 32 mm, 33 mm, 34 mm, or 35 mm. L1 and/or L2 may be 20-200% a magnitude of L3, and in many cases, at least 75-125%, 100-150%, or 50-75% of a magnitude of L3 or greater than a magnitude of L3. It will be recognized that occlusion device 100, 200, 300 may be selected for use in a procedure based on its dimensional closeness to geometries of a patient's anatomy, for example, as determined based on imaging. Various lengths of L1, L2, and/or L3 may be determined so as to effectively span a pyloric sphincter, extend a length deemed by a practitioner, medical professional, or automated system to be sufficient to allow for tissue ingrowth along respective retention member 102, 104, or any combination thereof.

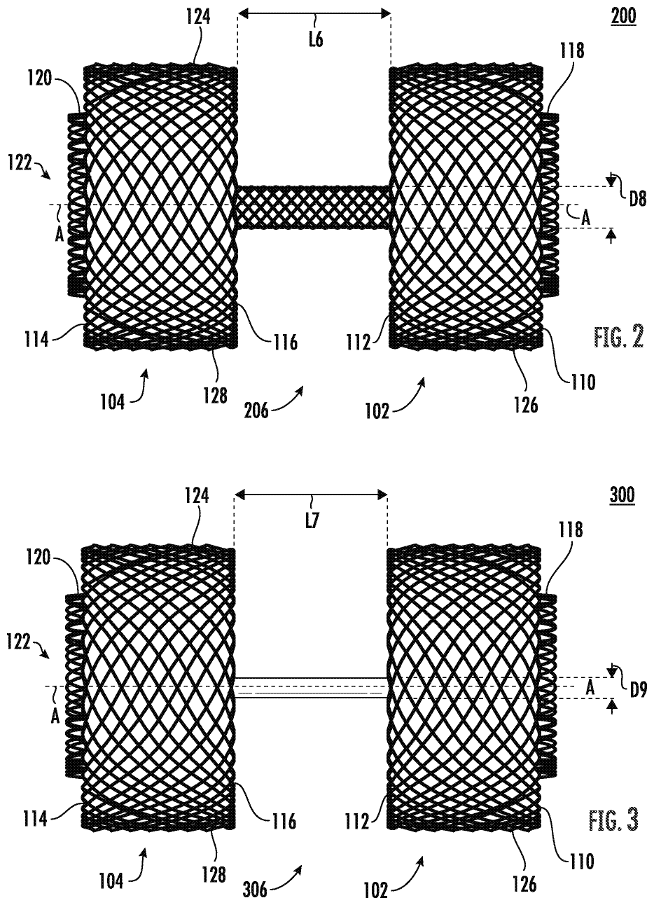

FIG. 2 illustrates an alternative embodiment, which, for the sake of simplicity, is described and illustrated with various components as described with respect to FIG. 1. However, an occlusion device 200 includes a saddle region 206 of length “L6” extending between first and second retention members 102, 104. Similarly to saddle region 106 of occlusion device 100, saddle region 206 may be continuously formed with first and second retention members 102, 104, for example, of woven filament 124. Dissimilarly to saddle region 106, saddle region 206 is a cylindrical saddle region comprising a substantially constant diameter “D8” along its full longitudinal length. In many embodiments, saddle region 206 may comprise a small enough diameter D8 so as to not continuously contact spanned tissue orifice 506, as illustrated in FIG. 5B (e.g., pyloric sphincter). Saddle region 206 may define lumen 122 and/or an outer surface of the elongate body extending a full length between the first and the second retention members 102, 104. Embodiments are not limited herein.

Each of saddle regions 106 and 206 may present various benefits. For example, saddle region 106 may provide selective flexibility along its length based on its diameter width, and in many cases may provide increased flexibility and/or deflectability at narrow portion 108, enabling it to conform to relative motion between spanned body lumens (not shown). Saddle region 206 may provide more consistent mechanical strength along its full axial/longitudinal length L6. Additionally, or alternatively, saddle regions 106 and 206 may differently accommodate a geometry of apposed tissue (not shown).

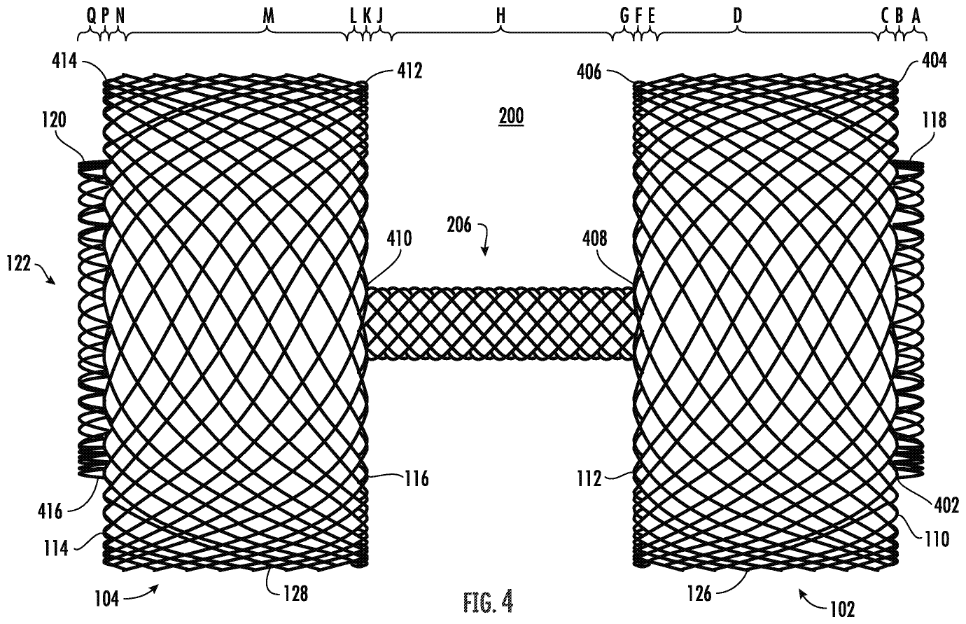

As an additional embodiment FIG. 3 shows an occlusion device 300, which is further described and illustrated with various elements as described in FIG. 1 for the sake of simplicity. Occlusion device 300 includes a saddle region 306, which is a continuous cylindrical bar of length “L7.” Saddle region 306 may be formed from a metal or polymer, and may be formed continuously with, welded to, glued to, or otherwise coupled with first and second retention members 102, 104. In some embodiments, saddle region 306 may be a single extruded member. Saddle region 306 may define lumen 122 and/or an outer surface of the elongate body extending a full length between the first and the second retention members 102, 104. In some embodiments, saddle region 306 may be formed of a woven braid such as saddle region 206, but not define lumen 122, or define lumen 122 so as to have a diameter of approximately 0.0 mm, thereby forming a barbell shape. Embodiments are not limited herein.

Lumen 122 may extend longitudinally through saddle region 306 (e.g., along axis A-A), or saddle region 306 may comprise a solid member.

Saddle region 306 may have less flexibility, or greater rigidity, than saddle region 106 or saddle region 206. It will be understood that less flexibility, or more rigidity, in a saddle region 106, 206, 306 may allow for less respective deflection as a result of motility of surrounding tissue(s). Accordingly, coupled retention members 102, 104 may contribute to greater aggravation and/or abrasion of apposed tissue(s) when coupled via saddle region 306 as opposed to saddle region 106, 206, thereby encouraging a greater growth response of the tissue(s) (not shown). Similarly, saddle region 206 may have less flexibility, or more rigidity, than saddle region 206.

Various embodiments may include sufficiently rigid first retention member 102, saddle region 106, 206, 306, and/or second retention member 104 so as to lodge within a body lumen (not shown) or across multiple body lumens (e.g., as illustrated in FIG. 5B). In embodiments where saddle region 306 comprises a solid member, the occlusion device 300 may particularly be designed to not conform to a surrounding tissue (e.g., saddle region 306 may not conform to a pyloric sphincter and/or a retention member 102, 104 may not conform to an apposed pyloric antrum) in order to allow for some limited flow alongside saddle region 306 (e.g., which may leak around or through an uncovered portion of the respective retention member).

In various embodiments, one or both of retention member 102, 104 and/or lip 118, 120 may not define lumen 122 (e.g., lumen 122 may not extend all the way through respective occlusion device 100, 200, 300). Some limited flow may still leak around or through an uncovered portion of the respective retention member, but at a reduced rate with respect to designs defining lumen 122 as extending through an entire length of occlusion device 100, 200, 300.

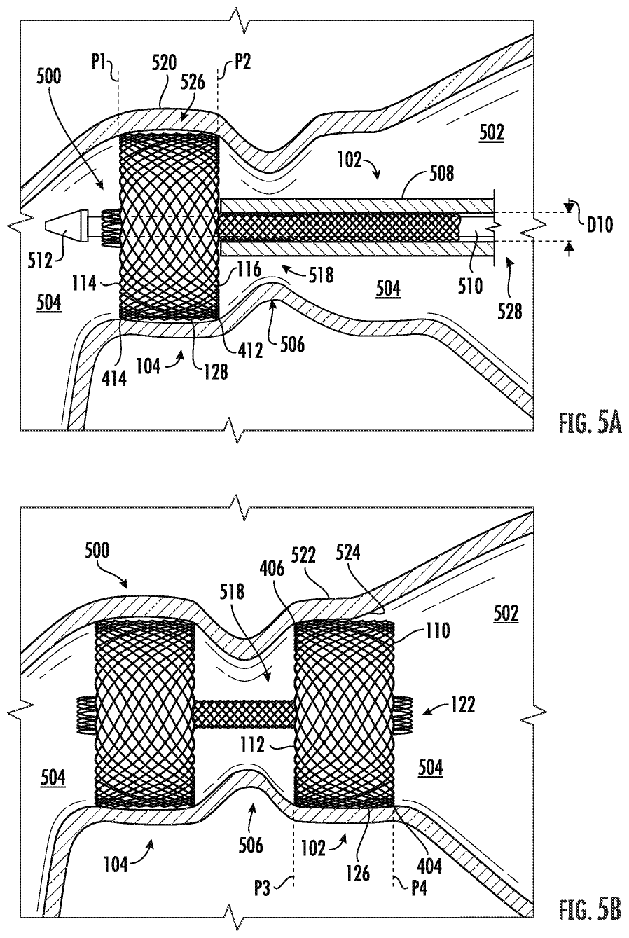

Any or all elements of occlusion devices 100, 200, 300 may comprise a cover or coating. For example, FIG. 4 illustrates occlusion device 200 as described above, labeled with various regions “A” “B,” “C,” “D,” “E,” “F,” “G,” “H,” “J,” “K,” “L,” “M,” “N,” “I′,” and “Q,” collectively referred to as regions “A-Q.” Any combination of regions A-Q may comprise a cover or be uncovered. While occlusion device 200 is referenced with respect to FIG. 4 for the sake of simplicity, it will be understood that regions A-Q may alternatively, or additionally, apply to occlusion devices 100 and/or 300. Embodiments are not limited in this context.

Each region A-Q may present various benefits in comprising and/or in not comprising a cover. For example, a cover may reduce, limit, or otherwise prevent tissue ingrowth along a respective region of a medical device. In some embodiments, a cover may increase a rigidity of or otherwise contribute a retentive strength of a respective region of a medical device. It is presently contemplated that strips, sheaths, or segments of cover may be applied selectively to various regions A-Q of occlusion device 200 in order to provide respective benefits.

Regions A and Q respectively refer to surfaces forming lips 118 and 120. Regions B and P respectively refer to surfaces forming outer walls 110 and 114. Regions C and N respectively refer to surfaces of first and second retention members 102, 104 adjacent to outer walls 110 and 114.

Regions F and K respectively refer to surfaces forming inner walls 112 and 116. Regions E and L respectively refer to surfaces adjacent to inner walls 112 and 116 of first and second retention members 102, 104. Region D extends along first retention member 102 between region C and region E. Region M extends along second retention member 104 between region L and region N.

Regions G and J respectively refer to surfaces along saddle region 206 adjacent to inner walls 112, 116. Region H extends along saddle region 206 between region G and region J.

A cover may increase a stiffness of a covered portion of occlusion device 200 by holding together a pitch of woven filament 124 and/or by applying a countering tension during deformation of occlusion device 200. It will be understood that, in particular, covering of adjacent regions may provide additional resistance of respective covered surfaces to deflection, deformation, or other movement with respect to each other. In various illustrative embodiments, covering of regions A and B may support an edge or interface 402 between lip 118 and outer wall 110. Covering of regions B and C may support an interface 404 between outer wall 110 and outwardly circumferential wall 126. An interface 406 between outwardly circumferential wall 126 and inner wall 112 may be supported by covering regions E and F. Covering of regions F and G may support an interface 408. Covering of regions J and K may support an interface 410. An interface 412 between inner wall 116 and outwardly circumferential wall 128 may be supported by covering regions K and L. Covering of regions N and P may support an interface 414 between outwardly circumferential wall 128 and outer wall 114. Covering of regions P and Q may support an interface 416 between outer wall 114 and lip 120.

Uncovered regions A-Q of occlusion device 200 may, however, comprise a rougher surface which may cause greater abrasion to an apposed tissue, and/or openings in pitch of woven filament 124 which may permit respective tissue ingrowth. In many embodiments, interfaces 404, 406, 412, and/or 414 may be left uncovered in order to generate a heightened growth response in apposed tissue (not shown).

It is further contemplated that any or each of the regions A-Q may comprise multiple portions which have or do not have a cover. Embodiments are not limited in this context.

FIGS. 5A-5B illustrate an illustrative delivery system and methods relating to medical devices as described herein. In particular, occlusion device 500 may comprise one or more similarities to occlusion devices 100, 200, 300 as described above. Occlusion device 500 is illustrated similarly as occlusion device 200, but similar and/or equivalent methods and devices may be used with other occlusion devices as disclosed herein. For example, saddle region 518 may be a saddle region 106, 206, 306, or other member extending between first and second retention members 102, 104. Embodiments are not limited in this context.

In use and by way of example, occlusion device 500 may be disposed in a first constrained configuration between an outer sheath 508 and an inner member 510 of a delivery system 528. For example, in a first constrained configuration, one or more of a first retention member 102, second retention member 104, and saddle region 518 may be restricted to an outer diameter “D10,” which in many embodiments may be the inner diameter of outer sheath 508. D10 may be less than one or more of D1-D9 as described above, so that occlusion device 500 may have a smaller outer diameter in the first constrained configuration than in a second unconstrained configuration. In some embodiments, inner member 510 may be a guidewire.

A tissue penetrating tip 512 may be coupled to inner member 510. In many embodiments, tissue penetrating tip 512 may include a point, knife, needle, electrocautery tip, or other element useful for extending delivery system 528 through one or more body lumens and/or tissues. In several embodiments, tissue penetrating tip 512 may include a camera or other visualization tool (not shown).

Tissue penetrating tip 512 may be advanced through a first body lumen 502 and into a second body lumen 504. In many embodiments, first body lumen 502 may be a pyloric antrum and second body lumen 504 may be a duodenum. First retention member 102 thus may function as a gastric extension of occlusion device 500. The occlusion device 500 may then be distally advanced with respect to outer sheath 508 such that outer wall 114 expands to a second unconstrained configuration at a predetermined position “P1,” outer sheath 508 may be proximally retracted with respect to occlusion device 500 such that inner wall 116 expands to a second unconstrained configuration at predetermined position “P2,” or both, such that second retention member 104 expands to a second unconstrained configuration, as shown in FIG. 5A. In many embodiments, outwardly circumferential wall 128 may be configured to appose tissue wall 520 defining second body lumen 504 when in the second unconstrained configuration. In particular, one or both of interfaces 412, 414 as described above with respect to FIG. 4 may be configured to appose tissue wall 520 when in the second unconstrained configuration. One or more of interfaces 412, 414 or outwardly circumferential wall 128 may be configured to expand or otherwise apply pressure to tissue wall 520 when in the second unconstrained configuration.

Occlusion device 500 may then be distally advanced beyond the outer sheath 508 such that inner wall 112 expands to a second unconstrained configuration at position “P3,” outer sheath 508 may be proximally retracted beyond occlusion device 500 such that outer wall 110 expands to a second unconstrained configuration at position “P4,” or both, and that thereby first retention member 102 expands to a second unconstrained configuration as shown in FIG. 5B. In many embodiments, one or more of interfaces 404, 406, or outwardly circumferential wall 126 as described above may be configured to appose tissue wall of first body lumen 502 in the second configuration.

In many embodiments, one or more of interfaces 404, 406, or outwardly circumferential wall 126 may be configured to expand or otherwise apply pressure to tissue wall 522 (e.g., pyloric antrum) when in the second unconstrained configuration. However, in many embodiments at least an area 524 along one or more of interfaces 404, 406, or outwardly circumferential wall 126 may not be configured to conform to a shape of tissue wall 522. In particular, first retention member 102 may not be configured or formed similarly to the shape of tissue wall 522, leaving a space of tissue 522 adjacent to area 524 unapposed by first retention member 102. Motility of body lumen 502 may thus irritate and/or contribute to some minor aggravation of tissue wall 522 by one or more of interfaces 404, 406, or outwardly circumferential wall 126, thereby causing a growth response of tissue wall 522. Length L1 of first retention member 102, as described above, may be configured to expose a sufficient portion of first retention member 102 to tissue wall 522 to accommodate tissue ingrowth thereinto. In many embodiments, L1 may be great enough so as to contribute to or otherwise create area 524 along first retention member 102 as described herein. Occlusion device 500 may thus be configured to cause an ingrowth of tissue wall 522 into one or more uncovered portions A-F of first retention member 102, as described above with respect to FIG. 4.

It will be understood that additionally, or alternatively, an area 526 along one or more interfaces 412, 414 or outwardly circumferential wall 128 may not be configured to conform to a shape of tissue wall 520. In particular, first retention member 104 may not be configured or formed similarly to the shape of tissue wall 520. Retention member 104 may thus be configured to irritate tissue wall 520 sufficiently to contribute to a growth response of tissue wall 520, particularly into one or more uncovered portions K-Q, as described above with respect to FIG. 4.

It will be understood that, while areas 524, 526 may be configured to not exactly correspond in shape to respective tissue walls 522, 520, one or more of areas 524, 526 may be closely aligned or otherwise adjacent to the tissue(s). Accordingly, the growth response of the respective tissue wall 520, 522 may reach and interact with area 526, 524 so as to substantially couple the occlusion device 500 to the tissue wall 520, 522, reducing a risk of migration of occlusion device 500.

In the second unconstrained configuration, saddle region 518 may be configured to span a distance between first and second body lumens 502, 504, and in particular, tissue orifice 506. In many embodiments, tissue orifice 506 may be a pyloric sphincter. As may be seen in FIGS. 5A-5B, one or more of saddle region 518 or its adjacent inner walls 112, 116 may not be configured to conform to a shape of tissue orifice 506, which may increase a contact pressure of one or more of interfaces 406, 412 against apposed tissue walls 522, 520 based on a narrowing of tissue orifice 506 unsupported by saddle region 518. In many embodiments, saddle region 518 may be a cylindrical saddle region, which may achieve one or more of these benefits. The increased contact pressure may, in several embodiments, contribute to a decreased migration risk of occlusion device 500.

In some examples, occlusion device 500 may be positioned across tissue orifice 506 so as to form an imperfect fit with one or more of tissue walls 520, 522 (e.g., including one or more of areas 524, 526) so as to allow some fluid flow around first and second retention members 102, 104. Alternatively, in the method above, a separate instrument with a sharpened distal tip may be advanced along the path above and into the second body lumen 504 to create a path, a guidewire put in place and the separate instrument withdrawn over the guidewire, and a stent, according to the various embodiments described above, loaded on a delivery catheter inserted over the guidewire, and the stent then deployed according to the steps outlined above (not shown). After occlusion device 500 has moved from the first constrained to the second unconstrained configuration, inner member 510 may be proximally retracted through lumen 122 from occlusion device 500.

Referring now to FIGS. 6A-6F, one or more occlusion devices as described herein may have variously configured lips as described above, presently shown via side and end views. As may be seen in FIGS. 6A-6F, as well as in FIG. 1 with respect to lips 118, 120, a lip may comprise an approximately lateral extension of an elongate body from a respective retention member away from a saddle region. Various lips as described herein may allow for removal of a medical device by a method of applying a pulling force to the lip, such as via a grasping element (not shown). In some embodiments, lips as described herein may abut a tissue wall (not shown), thereby causing abrasion resulting in a growth response of the tissue wall. Thus, lips of various geometries and/or configurations may provide accordingly different interactions with tissues, benefits for manipulation of a respective medical device, or other strength. While FIGS. 6A-6F refer to lip 118 alone for the sake of brevity, it will be understood that description may individually correspond and/or equivalently apply to lip 120. Embodiments are not limited in this context.

Lip 118 may extend from outer wall 110 of first retention member 102, as described above. Various embodiments may include a lip 118 of a large diameter “D11” (e.g., as opposed to respective diameter D4 of second retention member 104), as shown in the side view of FIG. 6A and the end view of FIG. 6B. D11 may, for example, be within the inclusive range of 20-30 mm, and in many embodiments, 20-25 mm, or 21-23 mm. In some examples, D11 may be 60-100% the size of D4. Lip 118 may alternatively have a small diameter “D12” (e.g., as opposed to respective diameter D4), as shown in the side view of FIG. 6C and the end view of 6D. D12 may, for example, be less than 20 mm, and in many embodiments, 12-17 mm, 3-5 mm, or 1-2 mm. In some examples, D11 may be up to 60% the size of D4. Lumen 122 may thus provide a continuous path through a respective occlusion device, which in many embodiments may accommodate a fluid flow path in the second unconstrained configuration and/or an inner member 510 in the first constrained configuration, as described above with respect to FIGS. 5A-5B. In various embodiments, a retention member may comprise an inwardly directed lip (e.g., extending into lumen 122 towards a saddle region) (not shown). In some embodiments, a retention member may not comprise a lip, as shown of first retention member 102 in the side view of FIG. 6E and the end view of FIG. 6F. Outer wall 110 may comprise a closed end.

Some embodiments may include at least one further occlusion element or cap 700, 702, and/or 704 as illustrated in FIGS. 7A-7C. Cap 700, 702, and/or 704 may be formed of metal, polymer, or other material, and may be coupled to a lip 118, 120 as described above via weld, glue, suture, press fit, or other coupling. Cap 700, 702, and/or 706 may be separately or integrally formed with a respective lip 118, 120. A cap 700, 702, and/or 704 may have an outer diameter corresponding to an associated lip (e.g., lip 118, 120), which is illustrated as D3 as described above with respect to lip 120. In various embodiments, D3 may be the same as D11 or D12 as described above with respect to FIGS. 6A-D. Cap 700, 702, 704 may define lumen 122 with a respective diameter “D13,” “D14,” or “D15.” Each of D13, D14, D15 may accommodate a width of an inner member 510 as described above with respect to FIGS. 5A-5B, which in many embodiments, may be a guidewire. D13 may be a large cap lumen diameter, for example, greater than 2.5 mm beyond a diameter of inner member 510, and in many cases, 3-4 mm total. D14 may be a small cap lumen diameter, for example, up to 2.5 mm beyond a diameter of inner member 510, and in many cases, 1-2 mm total. Cap 704 may be a substantially closed cap, wherein D15 is, or is approximately, the width of a corresponding inner member 510 as described above.

Cap 700, 702, 704 may be removably coupled to a lip 118, 120, enabling adjustment of flow through an occlusion device as described herein by narrowing an effective minimal inner diameter D1 of the occlusion device. In some embodiments, a medical practitioner may selectively couple a cap 700, 702, 704 based on a desired adjustment of flow through an occlusion device as presently disclosed.

FIGS. 8A-8L illustrate alternative configurations for a cross-sectional profile of any of an inner wall 112 or 116 or an outer wall 110 or 114 found in either or both of the first or second retention member 102, 104 in the expanded configuration, as is described with respect to FIG. 1. For the sake of simplicity in the drawings, various elements as illustrated in FIG. 1, such as first and second retention member 102, 104, saddle region 106, lip 118, 120, inner wall 112, 116, outer wall 110, 114, longitudinal axis A-A and vertical planes B-B, C-C, may be referenced with respect to FIGS. 8A-8L without redrawing each possible permutation of the surfaces of FIGS. 8A-8L for such. Accordingly, for each of FIGS. 8A-8L, a first portion 802 is depicted as extending from a right side of the illustration into the respective wall. First portion 802 may be saddle region 106 or lip 118, 120, according to whether the illustrated wall corresponds to an inner wall 112, 116 or an outer wall 110, 114. The wall or surface may be depicted as extending into portion 808 on the left side of the illustration, which may represent either an outwardly circumferential wall 126 or an outwardly circumferential wall 128, based on whether the illustrated wall corresponds to a first or second retention member 102, 104. Any of inner wall 112, 116 or outer wall 110, 114 may comprise any of the illustrated configurations of FIGS. 8A-8L, and all combinations are presently contemplated.

Accordingly, embodiments may comprise multiple retention members with the same or with different configurations. For example, the first retention member 102 may comprise an inner wall 112 configurated as illustrated in FIG. 8A, and the second retention member 104 may comprise an inner wall 116 configurated as illustrated in FIG. 8C. Any combination of the described configurations is within the scope of the present disclosure.

Several embodiments may include one or more components for managing material flow therethrough. For example, various embodiments may include a valve, barrier member, funnel, tube, or other structure useful for restricting or managing a flow therethrough (not shown).

In various embodiments of a medical device, at least a portion of an inner wall of a retention member, an outer wall of a retention member, or both, may comprise at least one curved surface, straight surface, angle, inner radial circumference, outer radial circumference, surface extending or bending toward a vertical center plane of a saddle region or central region of the medical device, surface extending or bending away from the vertical center plane of the saddle or central region of the medical device, surface perpendicular to a longitudinal axis of the saddle region, surface parallel to the longitudinal axis of saddle region, or any combination thereof. It will be recognized that various surfaces extending in different directions may be more or less susceptible to directional deformations than each other. For example, a straight surface may transversely deform more easily than a curved surface but present greater longitudinal resistance to deformation (e.g., along an axis residing in the straight surface) than alternative configurations. Further, combinations of various surfaces (e.g., adjacent concave and convex surfaces) may contribute to an increased resistance of a medical device to migration, as migration may require a straightening (e.g., flattening, unfolding, compression, or the like) of an interface between the surfaces. Interfaces between various surfaces may comprise corners, creases, or angles, which may contribute to a more traumatic interface with a respectively apposed tissue wall than other embodiments (not shown). Accordingly, medical devices as described herein may comprise various surfaces and/or combinations thereof in order to provide increased and/or decreased retentive force, abrasion to tissue, correspondence to an anatomical geometry, or any combination thereof.

Inner wall 112, 116 and/or outer wall 110, 114 of either or both of first and second retention member 102, 104 may comprise substantially parallel surfaces or non-parallel surfaces, for example, with each comprising a different one of the examples of FIGS. 8A-F. In various embodiments, either or both of first and second retention member 102, 104 may comprise asymmetrical inward and outward wall surfaces. For example, a cross-sectional profile of the first retention member 102 or of the second retention member 104, or both, may be asymmetrical along longitudinal axis A-A of saddle region 106. In some embodiments, asymmetrical wall surfaces may each have a shape and/or strength, such as a retentive strength, and/or provide selective degrees of abrasion to an apposed tissue. Asymmetrical wall surfaces may thus respectively accommodate for interaction with tissues of appropriate geometries, tissue fragility, and/or tissue growth response.

A portion of a retention member with the greatest external diameter may form a portion 808 (e.g., circumferential wall) that may connect, couple, and/or extend between an inward wall surface and an outward wall surface of the retention member. For example, portion 808 may comprise a ridgeline along a curved meeting of the inner wall 112, 116 and the respective outer wall 110, 114, such as in FIG. 8B, or a portion 808 may comprise one or more portions substantially parallel to a plane containing the longitudinal axis A-A of the saddle region 106, such as in FIGS. 8A, 8C, 8D, and 8E. The portion 808 and a beginning of an inner wall 112, 116 of a first or second retention member 102, 104 and an end of a respective outer wall 110, 114 may not be longitudinally aligned with each other.

In various embodiments, either or both of first and second retention member 102, 104 may comprise at least one concave portion 804. A concave surface or portion of a retention member may bend toward a vertical center plane of the respective retention member 102, 104 along the longitudinal axis (e.g., vertical planes B-B and C-C). In various embodiments, either or both of first and second retention member 102, 104 may comprise at least one convex portion 806. In several embodiments, a convex surface or portion of a retention member may bend away from a vertical center plane of the respective retention member 102, 104 along the longitudinal axis (e.g., vertical planes B-B and C-C). Additionally, or alternatively, either or both of first and second retention member 102, 104 may comprise at least one substantially straight portion 812, which may extend towards, away from, or perpendicular to a vertical center plane of the respective retention member 102, 104 along the longitudinal axis (e.g., vertical planes B-B and C-C).

A surface of inner wall 112, 116 and/or outer wall 110, 114 of respective first and/or second retention members 102, 104, which is non-perpendicular to a plane comprising the longitudinal axis A-A (for example, with respect to inner wall 112, 116, extending generally toward saddle region 106 as described with respect to FIG. 1, or, with respect to outer wall 110, 114, extending generally away from saddle region 106) may increase a resistance of the respective retention member to deformation, thereby increasing a pull-out strength of the retention member with respect to an alternative configuration. Accordingly, a stent with retention members according to embodiments of the present disclosure may be subject to less migration than a respective alternative configuration.

In some embodiments, a projection of a surface of inner wall 112, 116 or outer wall 110, 114 non-perpendicular to a plane containing longitudinal axis A-A of the medical device may extend a distance of 0.0-5.0 mm, or 2.5-5.0 mm, or 0.0-10.0 mm, along a longitudinal axis A-A towards or away from vertical plane B-B, C-C of the respective first or second retention member 102, 104 (e.g., toward or away from the other retention member 102, 104) as illustrated in FIG. 1. However, in other embodiments in line with the present disclosure, a projection of a surface of inner wall 112, 116 or outer wall 110, 114 may extend a greater distance along the longitudinal axis A-A of the medical device.

FIGS. 8A-8L illustrate various examples of surfaces of inner wall 112, 116 or outer wall 110, 114, which may provide various retentive strengths and/or abrasive interfaces to apposed tissue walls (not shown). For example, surface 800 of FIG. 8A includes convex portion 806 which may present a relatively more traumatic (e.g., abrasive) surface for apposition with a tissue wall than alternative configurations. Surface 810 of FIG. 8B includes convex portion 806 which may present a less traumatic surface for tissue wall apposition than in surface 800, but which may maintain a higher retentive strength than alternative configurations due to a small interior angle of concave surface 804. Various benefits and geometries of surfaces of FIGS. 8A-8L are described below with respect to each figure.

While reference is made throughout description of FIGS. 8A-8L of surfaces extending into one another for the sake of simplicity, it will be understood that in non-limiting examples, surfaces may otherwise couple with, join to, or bend into each other, or the like.

For example, as illustrated of surface 800 in FIG. 8A, portion 802 may extend into portion 808 via concave portion 804 and convex portion 806. In particular, portion 802 may extend into concave portion 804, which may extend into convex portion 806. In some embodiments, convex portion 806 may comprise a radius of curvature one third to one half of a respective D4 or D7 of a first or second retention member 102, 104 as described above. As shown in FIG. 8A, concave portion 804 may comprise a radius of curvature smaller than that of convex portion 806, such as 0-3 mm, or 2-5 mm.

With respect to surface 810 of FIG. 8B, the portion 802 may extend into portion 808 via concave portion 804, substantially straight portion 812, and convex portion 806. The portion 802 may extend into concave portion 804, which may extend into substantially straight portion 812 and have a smaller radius of curvature relative to convex portion 806 (e.g., 2-7 mm, or 3-6 mm, or 4-5 mm). Substantially straight portion 812 extends at an angle of less than 90 degrees towards a vertical center plane of the appropriate first or second retention member 102, 104 (e.g., vertical planes B-B and C-C). The convex portion 806 extends between the substantially straight portion 812 and the portion 808 and may have a radius of curvature, for example, between 7 and 20 mm, and in many embodiments, 10-15 mm.

The portion 808 may comprise a portion parallel to a surface of the portion 802. In various embodiments, as illustrated in FIG. 8A, concave portion 804 may have a greater internal radius of curvature than convex portion 806. However, it will be recognized that alternative embodiments may include varying relative radii of curvature between portions. For example, FIG. 8B illustrates concave portion 804 with a smaller radius of curvature than convex portion 806 (e.g., 0-5 mm). One or more convex and/or concave surfaces may include the same or various angles, radii of curvature, lengths, other feature, or any combination thereof.

Surface 818 of FIG. 8C may present a less abrasive surface to tissue and a lower retentive strength than surface 810. For example, a retention member may comprise concave portion 804 and convex portion 806 as described with respect to FIG. 8A. However, convex portion 806 as illustrated in FIG. 8B may comprise a larger radius of curvature than as illustrated in FIG. 8A (e.g., 7-15 mm). In FIG. 8B, convex portion 806 may comprise varying radii of curvature along its length. In some embodiments, convex portion 806 or other portion may comprise a non-uniform and/or otherwise undulating curve.

In FIG. 8D, surface 820 is formed by a continuation of portion 802 into concave portion 804, here comprising an interior angle of 90 degrees, which extends into substantially straight portion 812 perpendicular to portion 802 (e.g., to longitudinal axis A-A). Substantially straight portion 812 extends into convex portion 806 comprising an interior angle of 90 degrees, which extends into portion 808. As shown in FIG. 8D, either or both of a concave portion 804 and convex portion 806 may comprise a small enough radius of curvature so as to result substantially in a corner or an edge (e.g., 0-3 mm), which may increase trauma to an apposed tissue as compared to a portion with a larger radius of curvature (not shown).

FIG. 8E illustrates another illustrative embodiment, in which surface 822 may comprise similar elements including portion 802, concave portion 804, substantially straight portion 812, convex portion 806, and portion 808 as described above. However, surface 822 of FIG. 8E may have a higher retentive strength than surface 820 while providing less traumatic interfaces for tissue apposition based on higher interior angles of concave surface 804 and/or convex surface 806. Concave portion 804 as illustrated in FIG. 8E comprises a larger radius of curvature than as illustrated in FIG. 8C, for example, and convex portion 806 includes a larger radius of curvature than as illustrated in FIG. 8A. In some embodiments, concave portion 804 and convex portion 806 may comprise relatively equal radii of curvature (e.g., 5-15 mm, or 7-12 mm).

Surface 824 of FIG. 8F may present a relatively lower retentive force and/or abrasion to apposed tissue than alternative configurations. Regarding surface 824, portion 802 extends into concave portion 804, which is similarly configured as in FIG. 8D. However, rather than extending into substantially straight portion 812, concave portion 804 extends directly into a convex portion 806 with a broad curve sweeping to a portion 808, which may be a ridgeline if the alternative wall of the same retention member is additionally a surface without a longitudinally extending portion 808. For example, first retention member 102 comprising each of outer wall 110 and inner wall 112 with surface 824 would comprise a bulbous surface with portion 808 being a radially outermost edge (not shown). Convex portion 806 may, in such embodiments, comprise a radius of curvature between 5 and 20 mm, and in many embodiments, 7-12 mm.

An additional example of an embodiment is illustrated in FIG. 8G. Surface 826 of FIG. 8G, by virtue of relatively smaller radii of curvature and/or interior angles of concave surface 804 and/or convex surface 806, may have higher retentive strength and/or a more abrasive interaction with apposed tissue than alternative embodiments. In surface 826, the concave portion 804 extends into substantially straight portion 812, which extends into convex portion 806, which may in turn extend into the portion 808. In some embodiments, a concave or convex portion 804, 806 may comprise a tight enough radius of curvature to comprise a crease, corner, or other fold (e.g., 0-3 mm, 0-2 mm, or 0-1 mm). The crease, corner, or other fold may be configured to straighten to move from a second configuration to a first configuration (e.g., in order to allow the elongate body to move from the second configuration to the first configuration).

FIG. 8H illustrates another illustrative surface 828, in which portion 802, concave portion 804, and portion 808 may comprise one or more similar elements and/or properties as corresponding portions described above. However, in FIG. 8H, convex portion 806 comprises a smaller radius of curvature than as illustrated in FIG. 8A (e.g., 0-2 mm), which may result in more trauma for a tissue apposed by convex portion 806. Surface 828 may have a lower retentive strength than surface 826 by virtue of a larger relative radius of curvature of concave surface 804, but have a more abrasive interaction with apposed tissue by virtue of its smaller interior angle and/or radius of curvature of convex surface 806.

Surface 830 in FIG. 8I includes portion 802, concave portion 804, substantially straight portion 812, and portion 808 with one or more similarities as corresponding portions described above. However, in FIG. 8I, convex portion 806 comprises a larger radius of curvature and concave portion 804 comprises a smaller radius of curvature than, for example, surface 828 as illustrated in FIG. 8H, which may result in less trauma for a tissue apposed by convex portion 806 (e.g., respective radii of curvature of 5-15 mm and 0-5 mm).

FIG. 8J illustrates an additional example of a surface 832, in which portion 802, concave portion 804, substantially straight portion 812, and portion 808 may comprise one or more similarities as corresponding portions described above, particularly with respect to FIG. 8D. However, in FIG. 8J, convex portion 806 comprises a much larger radius of curvature than as illustrated in FIG. 8D (e.g., 4-12 mm), which may result in less trauma for a tissue apposed by convex portion 806 than alternative configurations such as surface 820.

FIG. 8K shows surface 834, comprising similar portion 802, concave portion 804, convex portion 816, and portion 808 as described above. Concave portion 804, as illustrated in FIG. 8K, may comprise a larger radius of curvature than as illustrated in FIG. 8A. As illustrated in FIG. 8K, concave portion 804 may extend, or dip, into a narrower radius than the respective portion 802. In some embodiments, concave portion 804 or other portions may comprise a non-uniform and/or otherwise undulating curve.

With respect to surface 836 of FIG. 8L, portion 802, concave portion 804, substantially straight portion 812, convex portion 806, and portion 808 may include one or more similarities as described above, such as with respect to FIG. 8D. However, concave portion 804 and convex portion 806 each comprise interior angles of greater than 90 degrees. Convex portion 806, for example, may present a less traumatic tissue interface in FIG. 8L than in FIG. 8D. It will be understood that a retention member comprising axially inward and outward walls with surface 836 may be roughly hexagonal in shape.

While various portions are described with respect to FIGS. 8A-8L, it will be appreciated that various embodiments may comprise one or more similarities and/or differences from the illustrated examples. For example, a surface may comprise more or fewer portions, and any portion thereof may comprise at least one concave section, convex section, straight edge, or any combination thereof. A portion may extend toward a first or second end of the device, perpendicular to a surface of axis A-A, parallel to axis A-A, at another angle with respect to axis A-A, or any combination thereof. Furthermore, dimensions and/or orientations of any of the portions described with respect to FIGS. 8A-L may be applied to other portions described therewith, alternatively or in combination, or with portions and/or retention member profiles otherwise with the scope of the present disclosure.

In several embodiments, smaller radii of curvature and/or interior angles in portions may contribute to a higher retentive strength of the corresponding retention member. For example, the configuration illustrated in FIG. 8G may comprise a greater resistance to a deformation than the respective configuration of FIG. 8A or of FIG. 8L. Accordingly, embodiments may be configured in accordance with various retention member strength requirements as necessitated by at least one particular procedure, tissue, or other consideration.

While not presently illustrated, it is currently contemplated that various embodiments described herein may comprise one or more additional features designed to engage at least one tissue layer. For example, embodiments may include one or more textured surfaces, prongs, or other tissue-engaging elements along a first retention member, a second retention member, or any combination thereof.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

The foregoing discussion has broad application and has been presented for purposes of illustration and description and is not intended to limit the disclosure to the form or forms disclosed herein. It will be understood that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the term “comprises/comprising” does not exclude the presence of other elements or steps. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

1. A pyloric occlusion device, comprising:
an elongate body having a first configuration and a second configuration,
wherein the elongate body comprises a first retention member, a second retention member, and a cylindrical saddle region extending therebetween;
wherein the first retention member and the second retention member each include an inner wall, an outwardly circumferential wall forming a cylindrical portion, and an outer wall, a first interface connecting the cylindrical saddle region and the inner wall, a second interface connecting the inner wall and the outwardly circumferential wall, and a third interface connecting the outwardly circumferential wall and the outer wall;
wherein, in the second configuration:
the first and the second retention members are expanded to have greater diameters than in the first configuration,
at least one of the outwardly circumferential wall, the second interface, or the third interface of either the first retention member or the second retention member is configured to appose a pyloric antrum wall, and
the cylindrical saddle region is configured to span a pyloric sphincter.

2. The pyloric occlusion device of claim 1, wherein at least a portion of the outer wall is covered and one or both of the first and second interfaces is uncovered, or wherein one or both of the first and second interfaces is covered and at least a portion of the outer wall is uncovered on at least one of the first or second retention members.

3. The pyloric occlusion device of claim 1, wherein the first retention member, the second retention member, or both comprise a lip.

4. The pyloric occlusion device of claim 1, wherein each of the first and second retention members extends along a length at least 75% greater than a length of the cylindrical saddle region.

5. The pyloric occlusion device of claim 1, wherein the first interface, the second interface, the third interface, or any combination thereof of one of the first or second retention members comprises a corner or crease.

6. The pyloric occlusion device of claim 1, wherein the inner wall and the outer wall of at least one of the first or second retention members are substantially parallel.

7. The pyloric occlusion device of claim 1, wherein the inner wall and the outer wall of at least one of the first or second retention members are non-parallel.

8. The pyloric occlusion device of claim 1, wherein the outer wall, the inner wall, or both of the first retention member comprises a convex portion bending toward a vertical center plane of the first retention member.

9. The pyloric occlusion device of claim 1, wherein the outer wall, the inner wall, or both of the first retention member comprises a concave portion bending away from a vertical center plane of the first retention member.

10. The pyloric occlusion device of claim 1, wherein a diameter of the first retention member, a diameter of the second retention member, or both is 300-600% a diameter of the cylindrical saddle region.

11. The pyloric occlusion device of claim 1, wherein the cylindrical saddle region defines an outer surface of the elongate body extending a full length between the first and the second retention members.

12. A stent for occluding a pylorus, comprising:
an elongate body having a first configuration and a second configuration;
wherein, in the second configuration, the elongate body comprises a saddle region and a gastric extension with an inner wall, an outwardly circumferential wall, and an outer wall, a first interface connecting the inner wall and the outwardly circumferential wall, and the second interface connecting the outwardly circumferential wall and the outer wall;
wherein one or both of the first interface and the second interface includes a concave portion and a convex portion;
wherein the saddle region defines a lumen characterized by a sufficiently small diameter so as to limit flow therethrough; and
wherein the outwardly circumferential wall defines a cylindrical portion with a length sufficient to encourage tissue ingrowth therealong.

13. The stent of claim 12, wherein one or both of the first interface and the second interface includes a substantially straight portion.

14. The stent of claim 12, wherein at least a portion of the outer wall is covered and one or both of the first and second interfaces is uncovered, or wherein one or both of the first and second interfaces is covered and at least a portion of the outer wall is uncovered.

15. The stent of claim 12, wherein the first interface, the second interface, or both comprises a corner or crease.

16. The stent of claim 12, wherein the cylindrical portion extends along a length that is at least 75% of a length of the saddle region, wherein a diameter of the inner wall is at least 28 mm, wherein a diameter of the saddle region is less than 5 mm, or any combination thereof.

17. A pyloric occlusion device, comprising:
an elongate body having a constrained configuration and an unconstrained configuration, wherein, in the unconstrained configuration, the elongate body comprises:
a first retention member, a second retention member, and a saddle region extending therebetween,
wherein the saddle region is configured to span a pyloric sphincter;
wherein one of the first retention member or the second retention member comprises an inner wall, an outwardly circumferential wall, and an interface between the inner wall and the outwardly circumferential wall, wherein at least one of the outwardly circumferential wall or the interface is configured to appose a pyloric antrum wall; and
wherein the outwardly circumferential wall does not conform to a shape of the pyloric antrum wall.

18. The pyloric occlusion device of claim 17, wherein the interface comprises a corner or crease.

19. The pyloric occlusion device of claim 17, further comprising an outer wall and a second interface between the outer wall and the outwardly circumferential wall.

20. The pyloric occlusion device of claim 19, wherein at least a portion of the outwardly circumferential wall is covered and one or both of the interface and second interface is uncovered, or wherein one or both of the interface and second interface is covered and at least a portion of the outer wall is uncovered on at least one of the first or second retention members.
